(19) United States
(12) Patent Application Publication
(10) Pub. No.: US 2026/0364351 A1
(43) Pub. Date: Nov. 19, 2026

(54) MULTI-ELECTRODE STIMULATION THERAPY WITH SUB-THRESHOLD PRE-PULSES

(71) Applicant: BIOTRONIK SE & Co. KG, Berlin (DE)

(72) Inventors: Marcelo Baru, Tualatin, OR (US); Andrew B. Kibler, Lake Oswego, OR (US)

(21) Appl. No.: 19/475,789

(22) PCT Filed: Apr. 9, 2024
(86) PCT No.: PCT/EP2024/059536

§ 371 Date: Oct. 16, 2025

Related U.S. Application Data
(60) Provisional application No. 63/460,718, filed on Apr. 20, 2023.

(30) Foreign Application Priority Data
May 23, 2023 (EP) 23174872.4

(51) Int. Cl. A61N 1/36; A61N 1/05

(52) U.S. Cl. CPC A61N 1/36062; A61N 1/0551; A61N 1/36071; A61N 1/36139; A61N 1/36164; A61N 1/36175; A61N 1/36185

(57) ABSTRACT

A neurostimulation device (100), at least partially implantable into a patient, includes at least one lead including at least three electrodes and a pulse generator. The neurostimulation device (100) is configured to provide a neurostimulation pulse sequence including at least two phases. The at least two phases include at least one first phase with a pre-pulse adapted such that no evoked compound action potential, ECAP, is triggered, and the at least two phases further include at least one consecutive second phase with a stimulation pulse adapted such as to evoke an ECAP.

The present invention relates to a neurostimulation device, e.g., configured to provide spinal cord stimulation (SCS), respective methods, and computer programs.

Sub-perception spinal cord stimulation (SCS) therapy (delivered at an amplitude below perception threshold PT) is known to alleviate chronic neuropathic pain in patients. Sub-perception SCS typically utilizes high-frequency stimulation at hundreds of Hz or more and either stimulates continuously or in a duty-cycled manner to extend battery life.

HF-10, a high-frequency stimulation therapy at frequencies of around 10 kHz, yields an energy-costly sub-perception stimulation. These types of therapies presently run on rechargeable implantable pulse generators (IPGs). For some patients, however, a non-rechargeable IPG is preferred for ease of use. Sub-perception therapies such as HF-10 therapy, even if duty cycled, may not be suitable for implementation in an IPG powered from a primary cell which is expected to deliver therapy for more than 5 years without replacement.

Therapy provided in high-frequency bursts repeated at a lower frequency (e.g., BurstDR) enables implementing an IPG powered from a primary cell. BurstDR, however, suffers from poorer pain relief performance as evidenced by reported responder rates and levels of reduced pain relief in lower back and leg pain patients. It may also not always be sub-perception given that it utilizes rectangular stimulation pulses which have a recruitment order weighted toward activation of large diameter fibers associated with paresthesia.

Tonic stimulation is typically delivered at under 150 Hz is known to have a significantly lower energy consumption than therapies at higher frequencies. Typically, however, it cannot offer paresthesia-free therapy but can merely replace the patient's pain by another sensation associated with the induced paresthesia according to the gate control theory of pain.

Therefore, there is still a need to improve neurostimulation devices.

According to one aspect of the invention, a neurostimulation device may be provided which is at least partially implantable into a patient. The neurostimulation device may comprise at least one lead comprising at least three electrodes and a pulse generator. The neurostimulation device may further be configured to provide a neurostimulation pulse sequence comprising at least two phases. Therein, the at least two phases comprise at least one first phase with a pre-pulse adapted such that no evoked compound action potential (ECAP) is triggered. The at least two phases further comprise at least one consecutive second phase with a stimulation pulse adapted so as to evoke an ECAP.

According to an embodiment, the neurostimulation device may further be configured to provide a neurostimulation pulse sequence comprising at least two common-polarity phases.

This may yield various advantageous effects. Dorsal root (DR) fibers usually have a lower threshold than the generally targeted dorsal column (DC) fibers. However, it may be aspired to selectively target the DC fibers in order to provide paresthesia-free therapy. While it is therefore challenging to evoke ECAPs in the DC fibers but not the DR fibers, it may be achieved more easily by the exemplary neurostimulation device. Applying sub-threshold pre-pulses, which do not evoke an ECAP, may tune the current-distance relationship of axons offering the opportunity to selectively (de-)activate fibers. The exemplary neurostimulation may offer a high degree of freedom in doing so with its three electrodes which may allow changing the type and/or location of stimulation and thus to tailor the according neurostimulation to the specific patient-related requirements and therefore increase efficiency, reliability and safety of the neurostimulation therapy treatment. Providing at least three electrodes may yield a high flexibility in how the electrodes may be activated and/or deactivated in each of the phases to tailor the stimulation according to the planned therapy.

The pulse generator may be configured to be implantable or placed on the skin of the patient and operably coupled with the electrodes of the neurostimulation device, e.g., via percutaneous wires. The pulse generator may by controlled by, e.g., a remote device which may be a remote control specifically provided for the neurostimulation device, a mobile phone via an according application, a computer, or any other device capable of communicating with the neurostimulation device. Such remote device may comprise a user interface allowing the patient and/or a health professional to provide input, at least in part based on which at least one stimulation parameter associated with the SCS may be adjusted. Such parameter may relate to any parameter described herein. Furthermore, an external server may be connected to the neurostimulation device and/or the respective remote device to transmit signalling based on which the SCS may be adjusted.

Generally, any function described herein in reference to the neurostimulation device and/or any part thereof may be transferred to an according remote device configured to exchange respective signalling with the neurostimulation device.

The neurostimulation device may comprise at least one lead with electrodes configured to be implanted in the patient. Typically, the at least one lead is placed in the epidural space between the spinal cord and the vertebrae. The lead(s) may be directly connected to the pulse generator providing an electrical path to the electrodes. There may be one or multiple leads with the same or different number and/or configuration of electrodes. The leads may be elongated leads adjusted to be inserted into the epidural space. Generally, however, any other lead placement is possible. For example, the at least one lead may be oriented in any angle relative to the spinal cord.

Generally, any number of electrodes (operating as cathodes and/or anodes) referred to herein may be located on the same and/or on different leads of the neurostimulation device, independent of the lead placement, lead design, and/or the number of leads comprised in the neurostimulation device.

Each phase may comprise multiple concurrent pulses provided by the electrodes of the neurostimulation device. Depending on the polarity, i.e., depending on whether being an anodic pulse or a cathodic pulse, the pulse may either be a hyperpolarizing pulse or a depolarizing pulse, respectively. In order to evoke an ECAP, depolarizing pulses may be used. In order to increase or decrease the threshold for evoking an ECAP, (sub-threshold) depolarizing pre-pulses, or hyperpolarizing pre-pulses may be applied, respectively.

The at least three involved electrodes may be utilized to provide the pre-pulse in the at least one first phase and/or the stimulation pulse in the at least one second phase. In some examples, however, less than the at least three electrodes may be involved in providing the pre-pulse in the at least one first phase. Having at least three electrodes allows for changes of the configuration of the involved cathodes and anodes between phases that go far beyond simply switching cathode and anode in a neurostimulation device with only two electrodes. However, the concept for the selective stimulation of axons described herein may also generally be realized in a neurostimulation device with less than three, such as only two electrodes configured to provide a neurostimulation pulse sequence comprising at least two phases. For example, therein, at least one first phase provides least one pre-pulse which is adapted such that no ECAP is triggered. The at least two phases further comprise at least one consecutive second phase with a stimulation pulse adapted such as to evoke an ECAP. In such an exemplary neurostimulation device, the amplitude and/or other parameters associated with the applied neurostimulation pulses of the neurostimulation may be varied and the cathode and anode may switch positions between phases. Such exemplary neurostimulation device may for example tailor the provided neurostimulation via selective stimulation of axons by providing more than one first phase with at least one sub-threshold pre-pulse.

In some examples, the neurostimulation may further be configured to vary the electrodes providing the neurostimulation between the at least one first phase and the at least one second phase. This may increase the flexibility in the electrode configuration of the neurostimulation device and allow to provide SCS in different modes. These may be adjusted to the requirements given by the health state of the respective patient and thus higher success chances may be achieved.

In some examples, the number of involved electrodes may be increased or decreased between the at least one first phase and the at least one second phase. The involved electrodes of any of the phases may be electrodes placed adjacent to each other on the lead of the neurostimulation device and/or they may be not adjacent to each other. Any pattern of active and inactive electrodes may be chosen for the at least one first phase and the at least one second phase. The pattern for the at least one first and/or second phase may thus be optimized with respect to the aspired effect, e.g., the sub-threshold pre-depolarization and/or the stimulation of the targeted axons to evoke an ECAP.

In some examples, even when the involved electrodes change between two consecutive phases, the number of active electrodes involved in delivering pulses to the patient may remain the same between consecutive phases, wherein e.g. electrodes at different positions participate in the various phases.

As another example, also other parameters associated with the neurostimulation pulses may be adjusted. These parameters may include a pulse waveform, a time delay between the at least one first phase and the at least one second phase, the (cumulative) pulse intensity, and/or an internal frequency modulating the pulse, etc.

In some examples, the neurostimulation device may deliver a pulse sequence comprising at least two first phases. The neurostimulation device may, optionally, further be configured to vary the electrodes providing the neurostimulation and/or to vary the pulse amplitude between the at least two first phases. This may improve the efficiency of the overall tuning of the current-distance relationship of axons offering the opportunity to activate fibers with an improved selectivity. Providing multiple phases comprising sub-threshold pre-pulses may thus decrease paresthesia and increase patient satisfaction and the over-all therapy treatment. For example, the number of involved electrodes may be increased or decreased between the at least two first phases. The involved electrodes of any of the at least two first phases may be electrodes placed adjacent to each other on the lead of the neurostimulation device and/or they may be not adjacent to each other. Any pattern of active and inactive electrodes may be chosen for the at least two first phases. The pattern for the at least two first phases may thus be optimized with respect to the aspired effect, e.g., the sub-threshold pre-depolarization and/or the stimulation of the targeted axons to evoke a compound action potential (CAP). The amplitude/intensity of the sub-threshold pre-pulses of consecutive phases of the at least one first phase may be increased phase-by-phase as the threshold of the non-targeted neurons to evoke a CAP may be increased by the pre-pulses of the respective preceding phase, and the threshold of the targeted neurons may be decreased by the same. Thus, such a process may cause a stepwise increase of the threshold of non-targeted neurons until the targeted neurons are prepared sufficiently for selective stimulation by the stimulation pulse.

Again, for example, also other parameters associated with the neurostimulation pulses may be adjusted between the at least two first phases. These parameters may include a pulse waveform, a time delay between the at least one first phase and the at least one second phase, the cumulative pulse intensity, and/or an internal frequency modulating the pulse.

The at least two first phases of such exemplary embodiments may thus be adjusted to each other as well as to the at least one second phase. This adjustment may be performed based at least on part on patient feedback. Such feedback may be given to a health professional adjusting the neurostimulation and/or via, e.g., a remote device or any other user interface, directly to the neurostimulation device.

In an example, the neurostimulation device may further be configured to change at least one electrode from a cathode to an anode and/or from an anode to a cathode between at least two of the at least two phases. This may provide a further degree of freedom which may improve the over-all efficiency of the SCS. Further, it allows to deliver the stimulation pulses exactly where the most suitable pre-depolarization adjusted the current-distance relationship of the axons in the vicinity of (or very close to) the active electrodes. “Axons in the vicinity of active electrodes” may be those axons that are less than 20 mm, in particular less than 5 mm, away from the active electrodes. This may, in particular, be advantageous when the active electrodes are arranged in a configuration where the electrical field resembles that of a classical dipolar configuration.

For example, the neurostimulation may be applied in multiple first phases in which at least one cathode and at least one anode may be at different positions in each phase such that at least one electrode may function as a cathode in one phase and an anode in another phase. This may occur in both directions (from cathode to anode and from anode to cathode).

Additionally or alternatively, the at least one electrode may be changed from a cathode to an anode and/or from an anode to a cathode between (e.g. a last) first phase and a subsequent second phase.

In some examples, the neurostimulation device may further be configured such that the at least one pre-pulse and the at least one stimulation pulse are applied having common polarities. This may yield a safe operation of the neurostimulation device wherein no unbalanced charges remain in the system.

Such charge balancing may be realized by short-circuiting at least two of the electrodes of the neurostimulation device, and/or any other active and/or passive charge balancing mechanism. It may involve the same and/or other electrodes that are involved in providing pre-pulses and/or stimulation pulses to the patient.

In an example, the neurostimulation device may further be configured to provide, in the at least one first phase and/or in the at least one second phase, at least one pulse having a polarity opposed to the pre-pulse and/or the stimulation pulse. This may provide a passive charge balancing effect at any time during the SCS which may increase patient safety and comfort by preventing the accumulation of charges within the patient.

In some examples, the neurostimulation may further be configured to repeatedly apply the pulse sequence. It may further optionally short-circuit the electrodes in between the pulse sequences.

The repeated application of the pulse sequence may sustain the treatment effect over an extended time period. At the same time, the sequence may be shorter compared to the time during which the SCS treatment is delivered. Generally, independent of the repetition pattern of the pulse sequence, the time between two consecutive pulse sequences may be significantly longer, e.g., at least ten-times or at least 100-times longer than the time between (any) two consecutive phases of a pulse sequence.

For example, the repeated delivery of the pulse sequence may be set to last a predetermined time and/or number of repetitions. In some examples, it may also be set to run until stopped by the device itself and/or input from the patient and/or the health professional, for instance via a remote control.

The repetition may occur in a periodic and/or in an irregular pattern. The according repetition rate and/or repetition pattern may be adjusted to the patient's needs, and/or it may be adjusted to the different times of the day. E.g., SCS frequency may be decreased overnight compared to the treatment at day or it may be adjusted in any other pattern. There may be predetermined modes from which the patient may choose one, e.g., each predetermined mode may be adjusted to a potentially fluctuating health state of the patient. In a simple example, the patient may be free to choose between modes labelled as “low”, “medium”, and “high”, configured to provide SCS with an increasing repetition rate and/or an accordingly changing repetition pattern. Such settings may also be changed by a health professional, potentially even via a remote control.

Short-circuiting the electrodes together between the pulse sequences may provide a passive charge balancing mechanism that requires only little or no energy. This may pose an efficient tool to increase battery life and safety as potential charge accumulation is prevented. Short-circuiting may occur after every pulse sequence and/or in any other irregular pattern.

In some examples, the neurostimulation device may further be configured to repeat the pulse sequence at a frequency of less than 1.5 kHz. Preferably, the frequency may be in the range of 2-130 Hz or 20-60 Hz. Choosing a repetition rate of below 1.5 kHz may reduce the energy consumption which may allow to use non-rechargeable batteries which may then still have a sufficient lifetime. This may increase patient comfort and satisfaction.

For example, the repetition rate may be adjusted by the patient and/or the health professional, e.g., within a predetermined range.

Providing SCS at such low frequencies, e.g., so-called tonic frequencies, may use less energy than, for example, HF-10 neurostimulation. Typically, tonic neurostimulation causes paresthesia. However, in combination with sub-threshold pre-pulses tuning the current-distance relationship for the selective activation of neurons, low-frequency neurostimulation may be provided in a paresthesia-free way. Thus, some examples of the neurostimulation device may yield the advantages of tonic neurostimulation like the low energy consumption and the use of non-rechargeable batteries while preventing the unwanted side-effect of paresthesia.

In some examples, the neurostimulation device may, in at least one of the at least two phases, be configured to provide neurostimulation in the following exemplary configurations: Firstly, the at least three electrodes may be arranged in a pseudo-monopolar configuration, wherein at least two electrodes may be located in a vicinity (or very close) to each other, and wherein at least one return electrode may be located at a distance from each of the at least two electrodes that is larger than any distance between the at least two electrodes.

“Electrodes in a vicinity to each other” may be such electrodes whose distance to each other is shorter than to other/further electrodes (e.g. the return electrode). The distance/spacing between two (adjacent) electrodes may be less than 10 mm, in particular less than 5 mm, in particular between 5 mm and 2 mm, in particular 4 mm. Thus, electrodes in a vicinity to each other may be such electrodes with a distance/spacing to each other of less than 20 mm, in particular less than 10 mm, in particular less than 5 mm. The electrodes may have a length between 5 mm and 2 mm, in particular 3 mm. The above millimeter specifications are non-limiting examples.

In a second example, the at least three electrodes with adequate spacing may be arranged to produce a pseudo-dipolar field. In a third example, the at least three electrodes may be arranged in a guarded electrode configuration, wherein at least one electrode may be located in between at least two return electrodes.

For example, at least one anodic return electrode may be configured to produce the charge balancing pulses and/or at least one cathodic electrode may be configured to deliver the sub-threshold pre-pulses and/or the above-threshold stimulation pulses.

In an exemplary pseudo-monopolar configuration, a plurality of neighboring cathodes may be separated from an anodic return electrode by at least one inactive electrode when for example the electrodes are arrayed along the length axis of the same lead (e.g., at a regular distance to each other). The return electrode may, in another example also be located on another lead. The distance between the cathodic electrodes and the anodic return electrode may be for example at least twice, five-times or ten-times larger than the mutual distance and/or the maximum distance between any of the electrodes of the plurality of cathodic electrodes.

In an exemplary pseudo-dipolar configuration, when the electrodes are arrayed along the length axis of the same lead, the plurality of cathodic electrodes may comprise neighboring electrodes and the anodic return electrode may be adjacent to either end of the plurality of cathodic electrodes. Analogously, any other and/or comparable configuration for electrodes which are not lined-up along one line is possible.

In an exemplary guarded electrode configuration, when the electrodes are arrayed along the length axis of the same lead, the plurality of cathodic electrodes may comprise neighboring electrodes on the lead. On each side of the plurality of cathodic electrodes there may be an anodic return electrode. The anodes may be separated from the plurality of cathodes by at least one inactive electrode. The respective distances between one anode and the plurality of cathodes on either side may be identical and/or different. When cathodes are added and/or removed between phases, another electrode may be used as an anode in the respective next phase to maintain a constant distance between the at least one anode and the plurality of cathodes. This may also apply to any other configuration, e.g., those described herein.

Further configurations beyond these examples are possible. Further, these exemplary configurations may be combined such that within one phase, the electrodes of the same and/or different leads of the neurostimulation device may be activated in any of the configurations described herein and/or any combination thereof. Further, such configuration may be varied from phase to phase offering a high degree of freedom to optimize the delivery of sub-threshold pre-pulse(s), the above-threshold stimulation pulse(s), and/or the charge balancing pulse(s).

Generally, along each lead, different target areas may be stimulated by a respective group of electrodes in a configuration which may be adjusted to the aspired SCS and/or the targeted neurons.

While these exemplary configurations were described in reference to leads with electrodes arranged in a linear array along the length axis of the lead of the neurostimulation device, the concept may analogously apply to any other pattern in which electrodes may be arranged relative to each other. This may relate to one-dimensional, two-dimensional, and/or three-dimensional electrode arrays/patterns, such as circular paths, spirals, etc.

An exemplary neurostimulation device may further be configured to deactivate at least one electrode after at least one phase during each neurostimulation pulse sequence. This may have an advantageous focus effect such that the area targeted by the respective active electrodes reduces when the at least one electrode is deactivated after at least one phase.

For example, one may provide sub-threshold pre-pulses via n cathodes and reduce the number of cathodes by one per phase such that n cathodes are active in the initial first phase, n-1 cathodes are active in the subsequent first phase etc. In an example, in the last phase with only one active cathode, the stimulation pulse may then be provided by the remaining cathode in an area which is relatively small compared to the area targeted by the pre-pulses in any of the previous phases. The pattern in which the cathodes are deactivated may be adjusted in any way. For example, when neighboring initially active cathodes are arranged in a linear array with a first end and a second end, after each phase, one cathode at the first end may be deactivated such that the remaining cathode in the last phase may be the cathode at the second end. This concept may also apply to any non-linear array of electrodes.

In another exemplary neurostimulation device, at least one previously inactive electrode may be activated after at least one phase during each neurostimulation pulse sequence. This may have a defocusing effect such that the area targeted by the respective active electrodes increases when the at least one electrode is activated after at least one phase.

In a further example, the neurostimulation device may comprise a circuitry configured to record the ECAP and/or to determine a time delay between the at least one stimulation pulse and the ECAP. ECAPs may be particularly suitable measures to monitor the undesired response of non-targeted and desired response threshold of targeted neurons. Non-targeted neural responses may be identified by a response delay A, and targeted neurons identified with a response delay B, where B is different from A. Thus, the circuitry configured to deliver stimulation pulses and determine a time delay may be adjusted to achieve a desired evoked response signal associated with the targeted neurons and avoid a response signal associated with the non-targeted neurons.

Further, a typical signal associated with the ECAP and measured by the circuitry may exhibit an amplitude maximum shortly after the rise of the ECAP signal. This maximum may be suited to define a point in time at which the ECAP is detected and may be used to determine the time delay between the stimulation pulse provided by the neurostimulation device and the ECAP and thus the type of nerve stimulated. This may allow to verify that the intended nerve fiber type is actually stimulated.

In some examples, the neurostimulation device may further be configured to automatically adjust at least one neurostimulation parameter based at least in part on the recorded ECAP and/or the determined time delay. This may pose an efficient way of optimizing the SCS in an automated way which does not require input from a health professional or the patient and may be performed at any time, potentially even without the patient noticing.

For example, the time delay may provide information about the signal transmission speed in the fibers in which the measured ECAP was evoked. As the signal transmission speed is related to the diameter of the fibers being recruited, the delay time may provide insights into which types of fibers were addressed by the SCS. This offers a useful tool to re-adjust the neurostimulation in, e.g., a closed-loop configuration until the measured delay time and/or any other parameter associated with the ECAP matches for example a predetermined parameter range expected for ECAPs triggered by the aspired SCS. Generally, when for example the distance between the electrode providing the stimulation pulse generating the ECAP and the electrodes measuring the ECAP is known, the time delay and the known distance may be used to calculate the signal transmission speed in the fibers in which the ECAP was triggered.

In response to an unsuitable ECAP signal, for example the activated electrodes, their configuration, the amplitude of any of the involved pre-pulses, stimulation pulses, and/or balancing pulses may be adjusted in each phase. Further the number of phases, parameters associated with duty-cycling, etc. may be adjusted to optimize the SCS.

For example, the neurostimulation device may further be configured to deliver the pulses in a duty cycle mode. The duty cycle may comprise an active interval, preferably programmable between 1 s and 10 h, which defines the interval of the duty cycle in which the neurostimulation device repeatedly provides the pulse sequence via the at least three electrodes. The duty cycle may further comprise an inactive interval, preferably programmable between 1 s and 10 h, which defines the interval of the duty cycle in which the neurostimulation device does not provide any neurostimulation. This may be a simple and efficient way to save energy and/or to prevent habituation.

The active and the inactive interval may be varied as required to find a point at which energy consumption is low, but still sufficient SCS is provided to the patient. In some examples, SCS may only be duty-cycled in response to user and/or health professional input, e.g., depending on the current health condition of the patient.

According to a further aspect of the invention, a system is disclosed comprising the described neurostimulation device, and at least one external device. The neurostimulation device and the external device are configured to establish a communication connection for enabling bidirectional communication between the neurostimulation device and the external device. The external device is configured to receive stimulation parameter information from the neurostimulation device and transmit adjusted stimulation parameters to the neurostimulation device via the communication connection. According to an embodiment, the neurostimulation device is capable of measuring ECAP signals, wherein the neurostimulation device is configured to transmit information associated with the ECAP signals to the external device via the communication connection.

According to an aspect of the invention a method for operating a neurostimulation device with at least three electrodes and a pulse generator may be provided. The method may comprise providing a neurostimulation pulse sequence. The pulse sequence may comprise at least two phases, wherein the at least two phases comprise at least one first phase with a pre-pulse adapted such that no ECAP is triggered, and the at least two phases may further comprise at least one consecutive second phase with a stimulation pulse adapted such as to evoke an ECAP. Further, any other aspect of the invention described as features of the neurostimulation device and/or instructions of an according computer program may analogously be implemented as steps of an according method.

According to a further aspect of the invention, a computer program comprising instructions may be provided. When executed, it may cause a neurostimulation device with at least three electrodes and a pulse generator to provide a neurostimulation pulse sequence comprising at least two phases. The at least two phases may comprise at least one first phase with a pre-pulse adapted such that no evoked ECAP is triggered, and the at least two phases may further comprise at least one consecutive second phase with a stimulation pulse adapted such as to evoke an ECAP. Further, any other aspect of the invention described as features of the neurostimulation device and/or steps of a method may analogously be implemented as instructions of an according computer program.

Accordingly, in one or more exemplary embodiments, the functions described may be implemented in hardware, software, or any combination thereof. If implemented in software, the computer program may be stored on or encoded as one or more instructions or code on a computer-readable medium such as computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

The computer program may be adapted to be executed on a processor. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise. By way of example, any means described herein, or any portion of the means, or any combination of means may be implemented as a “processing system” that includes one or more processors.

The devices and methods are described in the description herein and illustrated in the accompanying drawings by various blocks, components, circuits, processes, algorithms, etc. These means may be implemented using electronic hardware, computer software, or any combination thereof. Whether such means are implemented as hardware or software depends upon the particular application and design constraints imposed on the overall system.

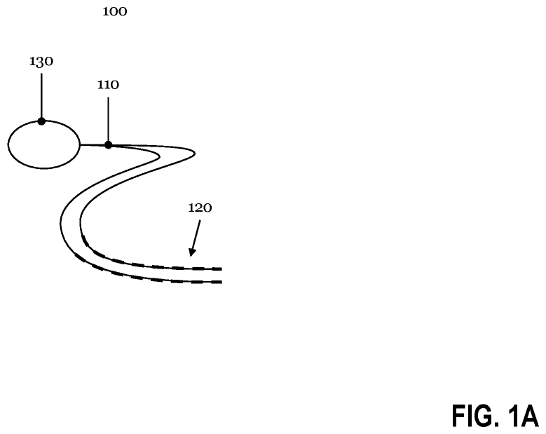

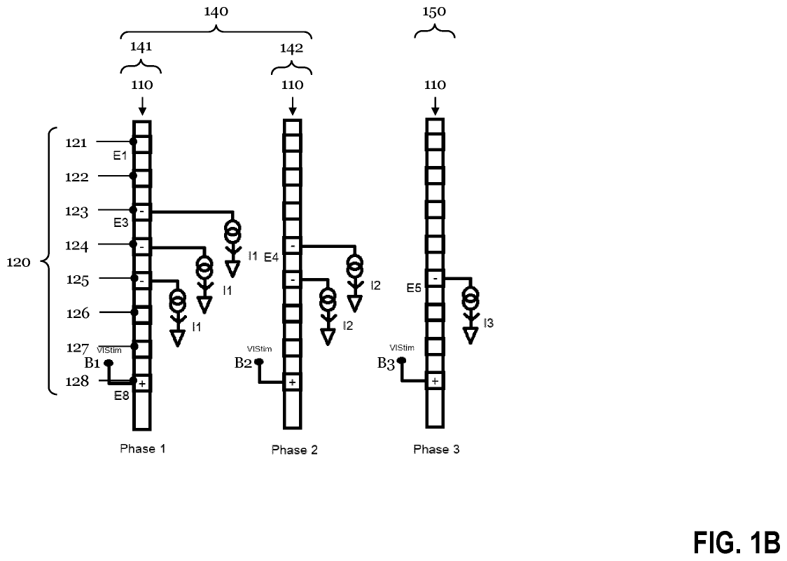

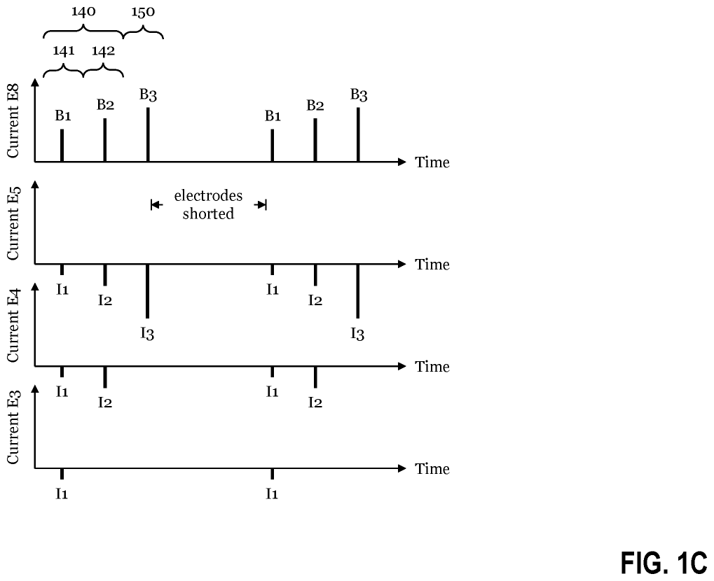

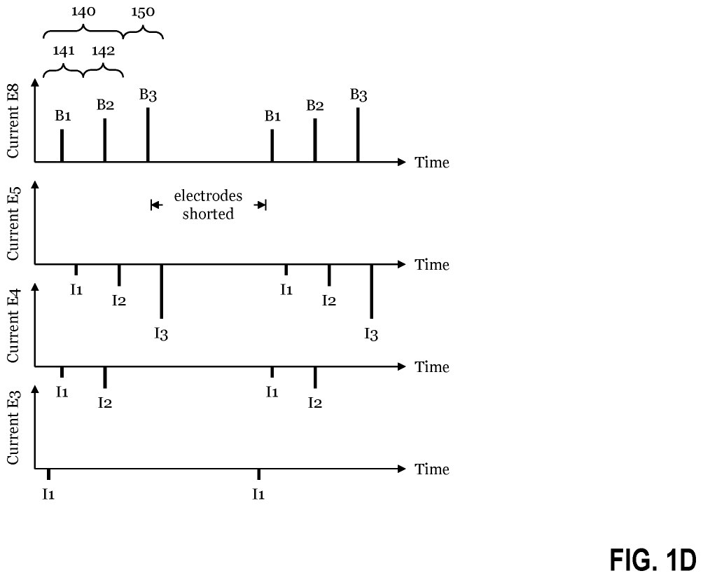

FIG. 1A Exemplary neurostimulation device with electrode leads and a pulse generator.

FIG. 1B Exemplary pseudo-monopolar embodiment of a neurostimulation device engaged in three phases to deliver neurostimulation, wherein one cathode is removed per phase.

FIG. 1C Schematic representation of temporal delivery of neurostimulation by the neurostimulation device of FIG. 1B

FIG. 1D Schematic representation of temporal delivery of neurostimulation by the neurostimulation device of FIG. 1B

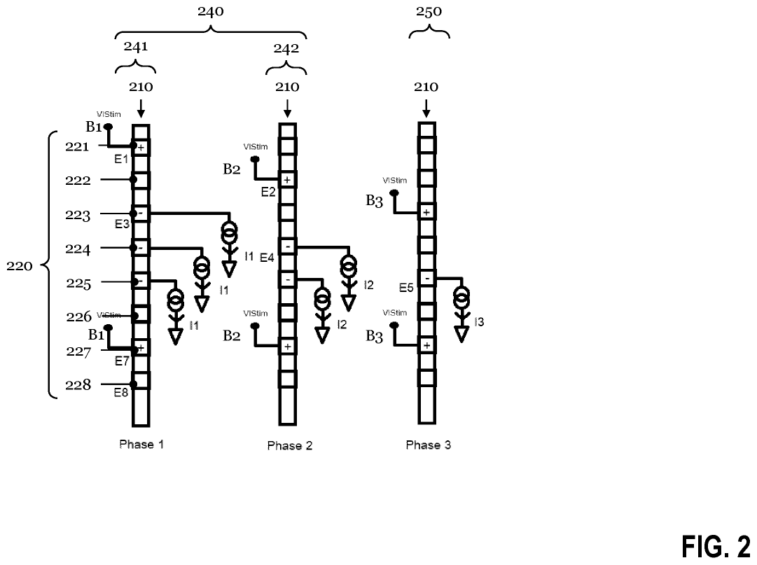

FIG. 2 Exemplary guarded cathode embodiment of a neurostimulation device engaged in three phases to deliver neurostimulation, wherein one cathode is removed per phase.

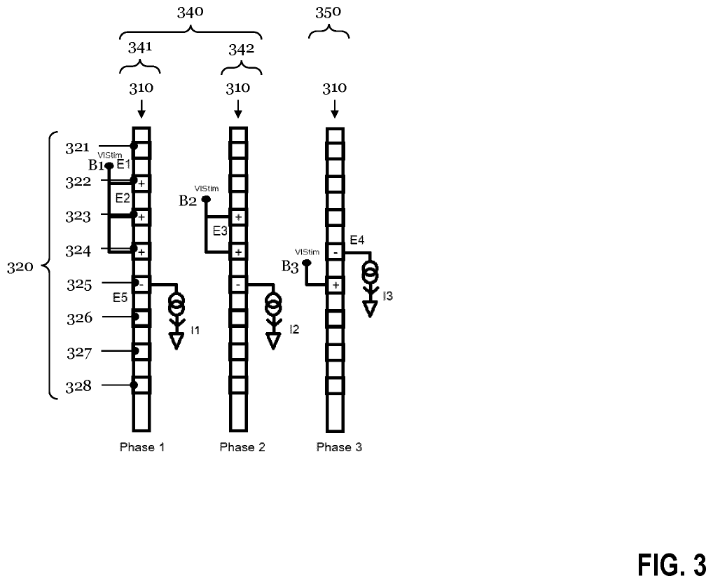

FIG. 3 Exemplary pseudo-dipolar embodiment of a neurostimulation device engaged in three phases to deliver neurostimulation, wherein one anode is removed per phase.

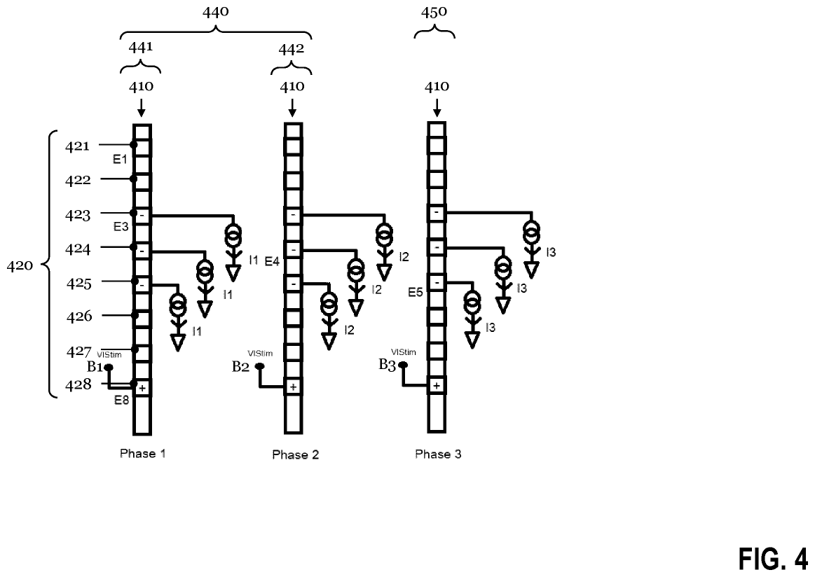

FIG. 4 Exemplary pseudo-monopolar embodiment of a neurostimulation device engaged in three phases to deliver neurostimulation, wherein the same electrodes are active in each phase.

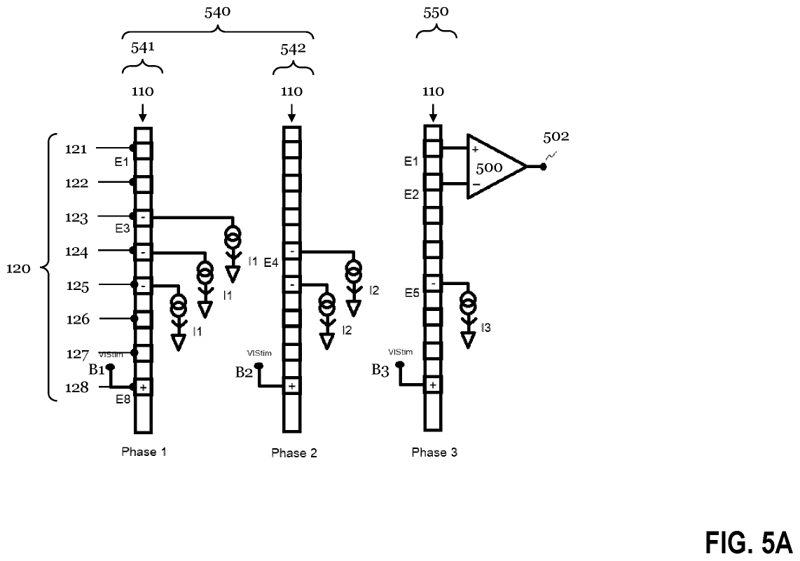

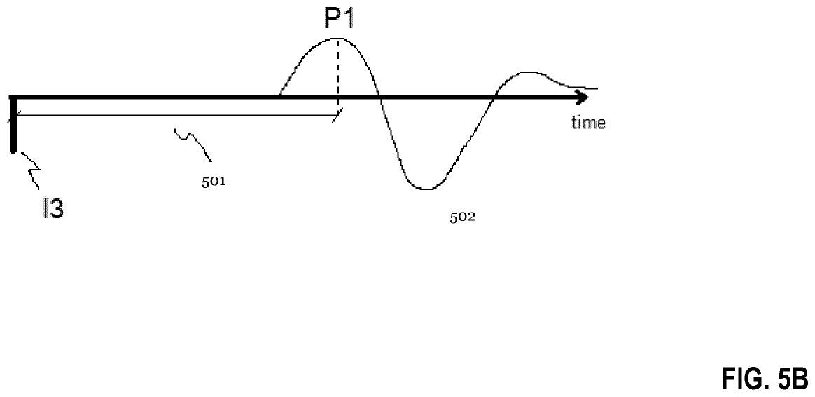

FIG. 5A Exemplary pseudo-monopolar embodiment of a neurostimulation device engaged in three phases to deliver neurostimulation, wherein one cathode is removed per phase and the neurostimulation device comprises front-end circuitry to sense ECAPs.

FIG. 5B Schematic representation of temporal delivery of a stimulation pulse by the neurostimulation device of FIG. 5A and the subsequent ECAP signal sensed by the front-end circuitry of the neurostimulation device.

In the following, phase 1 and phase 2 relate to two phases of the at least one first phase and phase 3 is one phase of the at least one second phase as described herein. However, this is only exemplary and more or less than first phases and/or more second phases may be used in other examples.

FIG. 1A shows an exemplary neurostimulation device 100 with two electrode leads 110 and a pulse generator 130. The neurostimulation device 100 may for example comprise two elongated leads 110 comprising a plurality of electrodes 120 each. The leads 110 of the neurostimulation device of FIG. 1A comprise eight electrodes 120 each which are lined up equidistantly at one end of the respective lead 110. At the other end, each lead 110 is connected to the pulse generator 130. The leads 110 may be at least partially flexible elongated leads 110 adjusted to be introduced into the epidural space between the spinal cord and the vertebrae. The leads 110 may be percutaneously implanted into the patient and placed in the vicinity of the axons to be stimulated.

FIG. 1B shows an exemplary arrangement of cathodes and anodes in an exemplary pseudo-monopolar configuration in an SCS delivered in three phases. The exemplary neurostimulation device 100 comprises a lead 110 with a plurality of electrodes 120, comprising eight programmable electrodes 121, 122, 123, 124, 125, 126, 127, 128 which may be active cathodes and/or anodes or inactive in any of the phases 141, 142 of the at least one first phase 140 and/or the at least one second phase 150. In other examples, a different number of electrodes 120 may be used. The electrodes 120 may be arranged in a linear fashion with a regular distance to each other.

In the first phase 141 of the at least one first phase 140, the three cathodes 123, 124, 125 deliver a current Il, each corresponding to sub-threshold stimulation. The anode 128 is connected to a voltage VIStim having a pulse of intensity B1 equal to the sum of I1 currents. Electrode 128 therefore acts as a return electrode which, in the pseudo-monopolar configuration of FIG. 1B, is located distantly from the cathodes 123 . . . 125 but still within the percutaneous electrode lead 110. In the example of FIG. 1B, two further (inactive) electrodes 126, 127 are located between anode 128 and the closest cathode 125.

In other examples, a different set of three (or more) electrodes 120 may be used as cathodes and/or a different electrode may be used as anode. Generally, however, the three or more cathodes may be next neighbors to each other and e.g. located relatively close to each other. The anode may in turn be located at a larger distance, e.g. it may not be a next neighbor to any of the three or more cathodes. For example, two, three or even more (inactive) electrodes may be located between the cathodes and the anode. When considering the configuration of FIG. 1B, electrode 128 may for example be the anode and the closest cathode may for example be electrode 123 or electrode 124.

The sub-threshold pre-pulses and stimulation pulse of the therapy of FIG. 1B pose a three-phase pulsing method wherein one cathode 123 . . . 125 is removed between consecutive phases. For example, after phase 141, electrode 123 is deactivated, and in phase 142 only electrodes 124 and 125 provide a current 12 which is returned again by anode 128 providing a pulse of intensity B2 equal to the sum of 12 currents. The cathodic pulses are again sub-threshold pulses. Generally, the cathode 123 . . . 125 most distant from the anode 128 may be deactivated. In an alternative embodiment, the cathode 123 . . . 125 closest to the anode 128 may be deactivated instead.

Lastly, in the at least one second phase 150, electrode 124 is deactivated such that the stimulation is further focused to the area of cathode 125, the only remaining active cathode. Electrode 125 provides an above-threshold cathodic and depolarizing stimulation pulse with an amplitude 13 returned again by an anodic pulse of intensity B3, provided by anode 128, resulting in dipole stimulation between electrodes 125 and 128. Generally, the remaining cathode(s) in the at least one second phase 150 may be that (those) closest to the anode.

In this example, in all three phases 141, 142, 150, the anode is electrode 128 which is distant to electrode 125. Generally, regarding this exemplary embodiment and/or other embodiments described herein, modes with any number of phases are possible, for example with two, four, five, or more phases. Like in the example of FIG. 1B one cathode may be removed between consecutive phases in an analogous or a different way than shown in FIG. 1B. Generally, in each phase 141, 142, 150, the cumulative intensity of cathodic pulses I1 . . . 13 may be controlled to be equal to the intensity of the corresponding return pulses B1 . . . B3.

Unlike Burst therapy approaches for SCS known from prior art, that employs a quiescent period in between stimulation pulses within a burst, the phases 141, 142, 150 of the exemplary therapy of FIG. 1B are preferably delivered without a pause. Altogether, it comprises two sub-threshold phases 141, 142 and one stimulation phase 150.

FIG. 1C shows the applied pulses per electrode 120 in time of the exemplary SCS of FIG. 1B delivered in three phases. In the first phase 141, all three cathodes 123, 124, 125 apply depolarizing pulses with an intensity I1 returned by the pulse with opposed polarity with an intensity B1 (equal to 3×I1) of the anode 128 provided at the same time. Phase 2 142 comprises two pre-depolarizing pulses, one provided by 124 and 125 each with an intensity I2. These are returned by the pulse B2 (equal to 2×I2) provided by electrode 128. In the third phase 150, electrodes 125 and 128 simultaneously provide pulses of equal amplitude (I3=B3) and opposed polarity. This pulse sequence comprising three phases 141, 142, 150 is repeated at a tonic frequency. As illustrated in FIG. 1C, the sub-perception therapy consists of pulse sequences of these three phases 141, 142, 150 repeated, e.g., at a tonic frequency, in a range of 2 Hz to 130 Hz, preferably at 40 Hz. In between phases 140, 150 sequence delivery, all participating electrodes 120 are shorted together to maintain charge balance (passive balance) minimizing power consumption. Generally, any characteristics described in reference to FIG. 1B/1C, like for example shorting the involved electrodes 120 for passive charge balancing, may not only be referred to the exemplary embodiment of FIG. 1B/1C but also to any other embodiment described herein.

FIG. 1D shows an alternative embodiment of the applied pulses per electrode 120 in time of the exemplary SCS of FIG. 1B delivered in three phases. In the first phase 141, all three cathodes 123, 124, 125 apply depolarizing pulses with an intensity I1 returned by the pulse with opposed polarity in a successive manner, with an intensity B1 (equal to 3×I1) of the anode 128 provided approximately at the time as the delivery of I1 of cathode 124. Phase 2 142 comprises two pre-depolarizing pulses, one provided by 124 and 125 each with an intensity I2 in a successive manner. These are returned by the pulse B2 (equal to 2×I2) provided by electrode 128 approximately at the same time as the delivery of I1 of cathode 124. In the third phase 150, electrodes 125 and 128 provide pulses of equal amplitude (I3=B3) and opposed polarity in a successive manner. This pulse sequence comprising three phases 141, 142, 150 is repeated at a tonic frequency. As illustrated in FIG. 1D, the sub-perception therapy consists of pulse sequences of these three phases 141, 142, 150 repeated, e.g., at a tonic frequency, in a range of 2 Hz to 130 Hz, preferably at 40 Hz. In between phases 140, 150 sequence delivery, all participating electrodes 120 are shorted together to maintain charge balance (passive balance) minimizing power consumption. Generally, any characteristics described in reference to FIG. 1B/1D, like for example shorting the involved electrodes 120 for passive charge balancing, may not only be referred to the exemplary embodiment of FIG. 1B/1D but also to any other embodiment described herein.

The stimulation currents I1-I3 for each phase may be independently programmable preferably in the range of 0.1 mA to 5.0 mA. Typically, stimulation currents I1 and I2 are equal to ⅓ stimulation current I3 or in a range of 15% to 50% thereof. In the exemplary SCS therapy, the amplitudes increase between the phases such that I1<I2<I3 and B1<B2<B3. The anode 128 may be connected to a programmable voltage VIStim generated from battery voltage, preferably in the range of 1.0 V to 16.0 V, that accommodates the overhead required for current stimulation. Different and varying-with-each-phase anode(s) 128 may be employed on the same lead 110 or different percutaneous leads 110.

FIG. 2 shows an alternative embodiment, wherein a lead 210 with a dual-anode-return via anodes 221 and 227 is employed (guarded-cathode configuration). The exemplary neurostimulation device of FIG. 2 may correspondingly comprise a lead 210 with a plurality of electrodes 220, comprising eight programmable electrodes 221, 222, 223, 224, 225, 226, 227, 228 which may be active cathodes and/or anodes or inactive in any of phases 241, 242 of at least one first phase 240 and/or at least one second phase 250 and generally be similar to those outlined with reference to FIG. 1B.

In phase 241, a sub-threshold pulse is applied by using adjacent electrodes 223, 224 and 225 as three cathodes. Two further electrodes 221 and 227 that are arranged symmetrically around the trio of cathodes 223, 224, 225, are used as two anodes that are connected with voltage VIStim. The anodes 221, 227 may be located at a distance to the trio 223, 224, 225, e.g. with one (inactive) electrode in between each of the anodes 221, 227 and the trio 223, 224, 225. Current pulses I1 are generated at each of the trio of cathodes 223, 224, 225.

Generally, also more than three cathodes may be used in other examples. Importantly, two further electrodes 221, 227, that are preferably arranged symmetrically around the cathodes 223, 224, 225 are used as anodes, and they may be nearest neighbors or next-to-nearest neighbors to the cathodes, for example. In some examples, the return anodes may be provided by the outermost electrodes of a lead (e.g., electrodes 221 and 228 in the example of FIG. 2).

In phase 242, another sub-threshold pulse is applied. An outermost one of the cathodes 223, 224, 225 is deactivated (in the example shown, cathode 223 is deactivated). The anode closest to the deactivated cathode (in the example shown, anode 221) is also deactivated, and instead another electrode is used to maintain symmetry of the “guarding” anodes (in the example shown, electrode 222 is used to replace anode 221). The anodes 222, 227 are connected to a voltage VIStim, and currents 12 are induced in each of cathode 224 and 225. In phase 250, an above-threshold pulse is applied. Only one of the electrodes remains as a cathode, namely electrode 225. That is, the cathode closest to the previously deactivated cathode is deactivated. Again to maintain symmetry of the “guarding” anodes, anode 222 is replaced by anode 223, such that two anodes 223 and 227 remain symmetrically arranged as next-to-nearest neighbors around the remaining cathode 225. Both anodes 223, 227 are connected to a voltage VIStim and a current 13 is induced in cathode 225 to provide an above threshold stimulation pulse.

Overall, one of the “guarding” anodes varies between phases 1-3 to maintain symmetry. Electrodes 221, 227 start as anodes in phase 1 241, switch to 222, 227 for phase 2, and end up as 223, 227 for phase 3. In phase 1 241, electrodes 223, 224, 225 function as cathodes in full analogy to the SCS therapy described in reference to FIG. 1A-C and the number of cathodes is then reduced by one per phase.

The “guarded cathode” embodiment of FIG. 2 may further reduce recruitment of undesired DR fibers. In phase 1 241, anodes 221 and 227 guard the cathodes 223, 224, 225; in phase 2 242, anodes 222 and 227 guard the cathodes 224 and 225. In phase 3 250, the at least one second phase 250, the only one cathode 225 is guarded by the anodes 223 and 227.

FIG. 3 shows a further exemplary embodiment in which pseudo-dipolar stimulation is employed. The exemplary neurostimulation device of FIG. 3 comprises a lead 310 with a plurality of electrodes 320, comprising eight programmable electrodes 321, 322, 323, 324, 325, 326, 327, 328 which may be active cathodes and/or anodes or inactive in any of phases 341, 342 of at least one first phase 340 and/or at least one second phase 350.

Two sub-threshold pre-pulses are provided in the phases 341 and 342. The pre-pulses comprise inverse polarity compared to the polarity of the pre-pulses of FIG. 1B.

The first sub-threshold pre-pulse phase 1 341 is generated by four active electrodes 322, 323, 324, 325, one cathode 325 and three anodes 322, 323, 324. The anodes may generally be nearest neighbors and in other examples more than four electrodes may be used as anodes, for example. The cathode 325 may generally also be provided by an electrode that is a nearest neighbor adjacent to the trio (or larger set) of anodes 322, 323, 324. Stimulation voltage VIStim may be applied to each of anodes 322, 323, 324 which return the sub-threshold pulse current of I1 applied at cathode 325.

For phase 2 342, which is the second sub-threshold pre-pulse, anode 322 is deactivated which is furthest from cathode 325, and phase 2 342 is a three-electrode primary phase. Generally, that anode that is furthest from the cathode may be removed. A sub-threshold current pulse 12 may be applied at cathode 325 returned by applying a corresponding voltage VIStim to anodes 323, 324.

For the final stimulation pulse (phase 3 350), again the remaining anode furthest from the cathode is removed (in the shown example, anode 323 is furthest from cathode 325 and thus removed). Moreover, the polarity of the remaining electrodes 324, 325 is reversed such that electrode 324 serves as cathode and electrode 325 as anode in this final phase 3 350. Overall, this results in dipole stimulation between electrodes 324 and 325. A stimulation voltage VIStim is applied to anode 325 and an above threshold current pulse I3 is applied at cathode 324.

While in this example, in the first two phases, the cathode was electrode 325, for the stimulation phase (phase 3 350), the configuration is flipped: Electrode 325 becomes the anode whereas electrode 324 is the cathode. In this configuration fibers with smaller diameter, in the vicinity of (or adjacent to) electrode 324, will be depolarized. This may account for the preferential sub-threshold depolarization in vicinity of (or adjacent to) electrode 324 in the exemplary pseudo-dipolar configuration shown in FIG. 3. In the exemplary embodiment, I1 and I2 may correspond to sub-threshold pulses and I3 to an above-threshold stimulation pulse.

FIG. 4 shows yet another possible embodiment comprising a lead 410 with a plurality of electrodes 420, comprising eight programmable electrodes 421, 422, 423, 424, 425, 426, 427, 428 which may be active cathodes and/or anodes or inactive in any of the phases 441, 442 of the at least one first phase 440 and/or the at least one second phase 450. Therein, the same cathodes 423, 424, 425 and anodes 428 participate in all three phases 441, 442, 450. In analogy to FIG. 1B, this embodiment exhibits a pseudo-monopolar configuration. Again, the cathodic pulses of phase 1 and 2 may be below-threshold depolarizing pulses with intensity I1 and 12, respectively. In phase three 450, the pulse amplitude I3 may correspond to a stimulating above-threshold pulse provided by all three cathodes 423, 424, 425 simultaneously. The anode 428 is connected to voltage VIStim for current circulation return.

The electrode configuration in phase 1 410 of FIG. 4 may generally be similar to that in phase 110 of FIG. 1B. The same electrode configuration may then be used in phases 2 and 3.

FIG. 5A shows a further exemplary embodiment in which a three-phasic stimulation is provided to the patient in phases 541, 542 of at least one first phase 540 and/or at least one second phase 550. It shows the configuration of FIG. 1B with an additional (front-end) circuitry 500 configured to sense ECAP signals 502. SCS may thus be delivered in a closed-loop fashion utilizing ECAP signal 502 sensing. Without losing generality, this is illustrated in FIG. 5A for the embodiment of FIG. 1B operating in closed loop. Also any other embodiment and/or electrode configuration may be combined with ECAP sensing functionality, as exemplarily shown in FIG. 5A by circuitry 500.

The ECAP signal 502 evoked by the last phase 550 of each pulse sequence may be sensed in distant electrodes (121 and 122) of the exemplary embodiment via the (front-end) circuitry 500. These electrodes may be located on an opposite side and/or end of lead 110 compared to anode 128. However, also other front-end configurations for ECAP signal 502 sensing in SCS may be employed. These may for example involve other electrodes than those shown in FIG. 1B that may be suitable to measure a propagation time of the evoked ECAP pulse.

FIG. 5B shows an exemplary temporal profile comprising the stimulation pulse 13 applied by the embodiment of FIG. 5A and the respective ECAP signal 502 with a first peak P1. The delay 501, from the start of the stimulation pulse I3 to the peak P1 of the ECAP signal 502 permits adjusting the stimulation parameters (I1, I2, I3 and the respective stimulation pulse widths) to recruit the desired fibers for sub-perception therapy. In such therapy large-diameter Aβ-fibers are preferably not to be recruited as these are associated with paresthesia. When recruited they will present the shortest delay 501. The sub-perception neurostimulation therapy disclosed herein will preferably avoid large-diameter fibers and this may be assessed via monitoring of the delay 501. Based on the sensed delay 501 and the known distance between the stimulating and the sensing electrodes, the signal transmission speed of the activated neurons may be determined. This information may be related to the type of neuron. In such closed-loop embodiments, the stimulation pattern may be adjusted based at least in part on the sensed ECAP 502, e.g., on the associated delay, until the sensed ECAP signal 502 indicates a well-adjusted SCS. This may be checked in reference to a predetermined set of reference data, e.g., comprising a range in which a delay 501 may be expected for a well-adjusted SCS.

While the embodiments described herein mostly comprise two phases 141, 142, 241, 242, 341, 342, 441, 442, 541, 542 of the at least one first phase 140, 240, 340, 440, 540 and one second phase 150, 250, 350, 450, 550, any other number of phases is possible. In detail, a stimulation pulse sequence may comprise less or more than two first phases 140, 240, 340, 440, 540 delivering sub-threshold pre-pulses and/or more than one above-threshold stimulation pulses applied in more than one phase of the at least one second phase 150, 250, 350, 450, 550. Any number of involved electrodes 120, 220, 320, 420 (cathodes and anodes) may be possible. Further, exemplary embodiments are possible in which multiple cathodes and/or multiple anodes provide pulses of different intensities within at least one phase.

1. A neurostimulation device (100), comprising:
(a) at least one lead (110) having at least three electrodes (120); and
(b) pulse generator (130);
wherein the neurostimulation device (100) is configured to provide a neurostimulation pulse sequence comprising at least two phases (140, 150); and
wherein the at least two phases comprise at least one first phase (140) with a pre-pulse adapted such that no evoked compound action potential, ECAP (502), is triggered, and the at least two phases (140, 150) further comprise at least one consecutive second phase (150) with a stimulation pulse adapted such as to evoke an ECAP (502).

2. The neurostimulation device (100) of claim 1, the neurostimulation device (100) being further configured to vary the electrodes (120) providing the neurostimulation between the at least one first phase (140) and the at least one second phase (150).

3. The neurostimulation device (100) of claim 1, wherein the pulse sequence comprises at least two first phases (141, 142), and wherein the neurostimulation device (100) is further configured to vary one or both of:
(i) the electrodes (120) providing the neurostimulation, or
(ii) the pulse amplitude between the at least two first phases (141, 142).

4. The neurostimulation device (100) of claim 1, the neurostimulation device (100) being further configured to perform one or both of the following:
(i) change at least one electrode (121-128) from a cathode to an anode between at least two of the at least two phases (140, 150), or
(ii) change at least one electrode (121-128) from an anode to a cathode between at least two of the at least two phases (140, 150).

5. The neurostimulation device (100) of claim 1, the neurostimulation device (100) being further configured such that the at least one pre-pulse and the at least one stimulation pulse are applied having common polarities.

6. The neurostimulation device (100) of claim 1, the neurostimulation device (100) being further configured to provide, in one or both of:
(i) the at least one first phase (140), or
(ii) the at least one second phase (150),
at least one pulse with a polarity opposed to one or both of:
(i) the pre-pulse, or
(ii) the stimulation pulse.

7. The neurostimulation device (100) of claim 1, the neurostimulation device (100) being further configured to repeatedly apply the pulse sequence.

8. The neurostimulation device (100) of claim 1, the neurostimulation device (100) being further configured to repeat the pulse sequence at a frequency of less than 1.5 kHz.

9. The neurostimulation device (100) of claim 1, wherein, in at least one of the at least two phases (140, 150), the at least three electrodes (120) are arranged in a pseudo-monopolar configuration, wherein at least two electrodes (123, 124, 125) are located in a vicinity to each other, and wherein at least one return electrode (128) is located at a distance from each of the at least two electrodes (123, 124, 125) that is larger than any distance between the at least two electrodes (123, 124, 125).

10. The neurostimulation device (100) of claim 1, the neurostimulation device (100) being further configured to deactivate at least one electrode (120) after at least one phase (141, 142) during each neurostimulation pulse sequence.

11. The neurostimulation device (100) of claim 1, further comprising circuitry configured to one or both of:
(i) record the ECAP (502), or
(i) determine a time delay between the at least one stimulation pulse and the ECAP (502).

12. The neurostimulation device (100) of claim 11, the neurostimulation device (100) being further configured to automatically adjust at least one neurostimulation parameter based at least in part on one or both of:
(i) the recorded ECAP (502), or
(ii) the determined time delay (501).

13. The neurostimulation device (100) of claim 1, the neurostimulation device (100) being further configured to deliver the pulses in a duty cycle mode, wherein the duty cycle comprises an active interval, which defines the interval of the duty cycle in which the neurostimulation device (100) repeatedly provides the pulse sequence via the at least three electrodes (120), and an inactive interval, which defines the interval of the duty cycle in which the neurostimulation device (100) does not provide any neurostimulation.

14. A method for operating a neurostimulation device (100) with at least three electrodes (120) and a pulse generator (130), the method comprising:
providing a neurostimulation pulse sequence comprising at least two phases (140, 150), wherein the at least two phases (140, 150) comprise at least one first phase (140) with a pre-pulse adapted such that no evoked compound action potential, ECAP (502), is triggered, and the at least two phases (140, 150) further comprise at least one consecutive second phase (150) with a stimulation pulse adapted such as to evoke an ECAP (502).

15. A computer program comprising instructions which, when executed causes a neurostimulation device (100) with at least three electrodes (120) and a pulse generator (130) to:
provide a neurostimulation pulse sequence comprising at least two phases (140, 150), wherein the at least two phases (140, 150) comprise at least one first phase (140) with a pre-pulse adapted such that no evoked compound action potential, ECAP (502), is triggered, and the at least two phases (140, 150) further comprise at least one consecutive second phase (150) with a stimulation pulse adapted such as to evoke an ECAP (502).

16. The neurostimulation device (100) of claim 7, the neurostimulation device (100) being further configured to short-circuit the electrodes (120) in between the pulse sequences.

17. The neurostimulation device (100) of claim 8, the neurostimulation device (100) being further configured to repeat the pulse sequence at a frequency of 2-130 Hz.

18. The neurostimulation device (100) of claim 1, wherein, in at least one of the at least two phases (140, 150), the at least three electrodes (322, 323, 324, 325) are arranged in a pseudo-dipolar configuration.

19. The neurostimulation device (100) of claim 1, wherein, in at least one of the at least two phases (140, 150), the at least three electrodes (221, 223, 224, 225, 227) are arranged in a guarded electrode configuration, wherein at least one electrode (223, 224, 225) is located in between at least two return electrodes (221, 227).

20. The neurostimulation device (100) of claim 13, wherein the active interval is programmable between 1 s and 10 h; and wherein the inactive interval is programmable between 1 s and 10 h.
